SEQUENCE LISTING (2 sequences)

SEQ ID NO: 1 - DNA, 23 bp, Artificial Sequence
  NPM1forward primer
ttaactctctggtggtagaatga

SEQ ID NO: 2 - DNA, 22 bp, Artificial Sequence
  NPM1 reverse primer
ctgaccaccgctactactatgt

(19) United States
(12) Patent Application Publication
(10) Pub. No.: US 2016/0305945 A1
(43) Pub. Date: Oct. 20, 2016

(54) Leukemic stem cell markers

(71) Applicant: Stichting VU-VUmc, Amsterdam (NL)

(72) Inventors: Gerrit Johan OSSENKOPPELE, Amsterdam (NL); Gerrit Jan SCHUURHUIS, Amsterdam (NL); Wendelien ZEIJLEMAKER, Amsterdam (NL)

(21) Appl. No.: 15/102,075

(22) PCT Filed: Dec. 2, 2014
(86) PCT No.: PCT/NL2014/050821

§ 371 Date: Jun. 6, 2016

(30) Foreign Application Priority Data
Dec. 6, 2013 (NL) 2011904

(51) Int. Cl. G01N 33/574; G01N 15/14; G01N 33/50

(52) U.S. Cl. CPC G01N 33/57426; G01N 33/5011; G01N 15/14; G01N 33/5073; G01N 2015/149; G01N 2015/1006

(57) ABSTRACT

The present invention relates to a method for identifying Leukemic stem cells (LSC), based on measurement of the presence of a specific set of markers. The invention further relates to a container for use in performing the method. Also provided is application of the method for predicting response to therapy and/or chance of relapse of Acute Myeloid Leukemia, and application of the method in the context of screening for compounds that specifically eradicate Leukemic cells and not benign cells.

### FIELD OF THE INVENTION

The present invention relates to a method for identifying Leukemic stem cells (LSC) in Acute Myeloid Leukemia (AML), based on measurement of the presence of a specific set of markers. The invention further relates to a container useful for performing the method. Also provided is application of the method for predicting response to therapy and/or chance of relapse of Acute Myeloid Leukemia, and application of the method in the context of screening for compounds that specifically eradicate LSC and not benign hematopoietic stem cells.

### BACKGROUND ART

Acute myeloid leukemia (AML), also known as acute myelogenous leukemia, is a heterogenous group of disorders that arise from the neoplastic transformation of a hematopoietic stem cell or progenitor cell of the bone marrow. Cells originating from such a transformed cell grow fast, and replace healthy cells. The disease is therefore characterized by an increase in the number and frequency of myeloid cells in the bone marrow and an arrest in their maturation.

The bone marrow, which normally helps the body fight infections, eventually will lose proper function. Consequently, people with AML become more susceptible to infections and are prone to bleeding because the number of healthy blood cells decrease. Other symptoms include easy bruising, bone pain or tenderness, fatigue, fever, heavy menstrual periods, pallor, shortness of breath, skin rash or lesion, and weight loss (Cecil Medicine. 23rd ed. Philadelphia, Pa.: Saunders Elsevier; 2007: chap 194).

Around 80% of adult AML patients (<60 years) achieve complete remission of the disease after intensive chemotherapy. However, in the majority of patients with AML who achieve a complete remission, the disease will recur within 3 years after diagnosis. In general, the prognosis of patients after relapse is poor and treatment options are unsatisfactory (Blood, 2010, Vol. 115, number 3, pp 453-474), resulting in five-year survival rates of around 30%-40%.

Relapse in AML likely originates from minimal residual disease (MRD) cells, at the basis of which are (therapy resistant) Leukemia initiating cells, further referred to as LSC. Consequently, quantifying these remaining LSC in AML, is of prognostic importance and correlates with the incidence of relapse (Leukemia 2007; 21: 1700-1707). In particular, quantifying MRD (remaining LSC) is a major issue in clinical management, e.g. for making the decision to clinically intervene, i.e. to identify patients with poor prognosis and re-allocate these to more intensive therapy regimens, while patients with less MRD (LSC) can be offered a less intensive treatment.

LSC in bone marrow can be identified and quantified by using cell surface antigen expression. It has been described that CD34+CD38− LSC (i.e. LSC that express CD34 but not CD38) may be most malignant and most resistant to therapy (Costello et al. Cancer Res. 2000; 60(16):4403-11). Zeijlemaker and Schuurhuis (2013) have reported that specific detection of LSC within the total CD34+CD38− compartment, containing both LSC and benign hematopoietic stem cells, referred to as HSC, in a bone marrow sample may be possible by making use, next to CD34 and CD38, of lineage markers including CD2, CD7, CD11b, CD13, CD15, CD19, CD22, CD33, CD56, and HLA DR, in combination with other markers including CLL-1 (also referred to as Clec12a), CD25, CD32, CD33, CD44, CD47, CD96, CD123, and TIM-3 (Chapter 6 in Leukemia, Guenova, ed., ISBN: 978-953-51-1127-6, InTech, 2013).

However, there is much heterogeneity in marker expression between different AML patients as well as within the LSC compartment within a single patient. Consequently, most of the markers detect LSC only in part of the patients, or even identify only part of the LSC within a single patient. Another problem is that in recent years it has become clear that LSC clones existing at diagnosis may disappear during disease progression while new clones expressing different marker(s) evolve. Due to this heterogeneity and instability of marker expression a broad spectrum of different markers must be used which is time- and bone marrow consuming and requires a large number of costly antibodies.

It is an object of the present invention to overcome at least one of the above-mentioned obstacles in the art.

### SUMMARY OF THE INVENTION

The present invention relates to a new broadly applicable method that can identify the cells that are thought to be at the origin of Acute Myeloid Leukemia: Leukemic stem cells (LSC). LSCs are present together with benign (normal) hematopoietic stem cells (HSCs) in the bone marrow and discrimination between the two is very instrumental in order to determine the chance of relapse of the disease and also for example to monitor the effects of new therapies with the aim to provide short-term outcome endpoints for such therapies.

The present invention now for the first time allows for adequate discrimination of LSC from HSC using a reduced number of markers that still deals with heterogeneity of marker expression on LSC among different patients and within single patients. In other words, the present selection of markers is effective in (substantially) all patients. In addition, markers identified as having unstable (varying) expression during disease progression were excluded. Furthermore, all markers of the present invention advantageously fit into one FACS tube for use in flow cytometry, by combining certain markers in one fluorochrome channel.

The method is applicable irrespective of the stage of the disease including diagnosis (although the method may also not be used for diagnosis). Importantly, it is now possible to assess LSC quantities in each laboratory equipped with standard flow cytometers in a routine setting, with neither knowledge required of the nature of the individual markers nor of other AML characteristics. These properties warrant the design of the method as a kit with antibodies for each marker according to the invention.

### DETAILED DESCRIPTION OF THE INVENTION

In a first aspect, the present disclosure provides for a method for identifying Leukemic stem cells (LSC), i.e. for recognizing cells as being LSC, the method comprising:
(a) providing a sample obtained from a subject, preferably a subject having or suspected of having AML (or myelodysplastic syndrome);
(b) measuring, for individual cells (e.g. of the stem cell fraction) of the sample, expression (or expression level) of a set of at most 25 markers, but more preferably at most 21, 20, 19, 18, 17, 16, 15, 14, or 13 markers, wherein the set comprises markers CD34, CD38, CD45, CD123, CD33, CLL-1, TIM-3, CD11b, and/or CD22;
(c) optionally, comparing the measured expression of the markers of the set with a reference, wherein preferably an individual cell is identified as an LSC if the cell is CD34+, CD38−, CD45+, and at least one of CD123+, CD33+, CLL-1+, TIM-3+, CD11b+, and CD22+.

Myolodysplastic Syndrome is a diverse collection of hematological (blood-related) medical conditions that involve ineffective production (or dysplasia) of the myeloid class of blood cells (Dorland's medical dictionary). The sample to be used is preferably a bone marrow sample or a peripheral blood sample. A bone marrow sample can be obtained by bone marrow biopsy or bone marrow aspiration, as well known to the skilled person and which can be carried by medically qualified personnel.

In a preferred embodiment, the set of markers further comprises markers CD45ra and/or CD44. Inclusion of these markers has certain further advantages. For example, CD45ra can be used to identify different maturation stages of CD34+CD38− LSC, and CD44 has the advantage that it can be used to identify nonspecific binding of antibodies against other markers. In addition, both markers contribute to the discrimination between LSC and HSC. The set of markers may also further comprise markers CD56 and/or CD7, which can further improve the ability of the method to discriminate between LSC and HSC. In a further or additional preference, the set of markers also includes CD2, CD15, and/or CD96.

Preferably, lower expression (or absence) of CD38, and higher expression (or presence) of CD34, CD45 and at least one (e.g. 2, 3, 4) further marker of the set, preferably as compared to a suitable reference, indicates that an individual cell is an LSC. On the other hand, lower expression (or absence) of CD38, higher expression (or presence) of CD34, CD45, in addition to absence of (higher) expression of the further markers of the set, preferably as compared to a suitable reference, indicates that an individual cell is a benign HSC.

In other words, or alternatively, an individual cell is identified as a Leukemic stem cell if the cell is CD34+, CD38−, CD45+, and at least one (e.g. 2, 3, or 4) of CD123+, CD33+, CLL-1+, TIM-3+, CD11b+, CD22+, and optionally CD45ra+, CD44+(+), CD56+, and CD7+ (and CD2+, CD15+, and CD96+). There is indication that an individual cell is a benign HSC if the cell is CD34+, CD38−, CD45+, and none (or <2, <1) of CD123+, CD33+, CLL-1+, TIM-3+, CD11b+, CD22+, and optionally CD45ra+, CD44+, CD56+, and CD7+ (and CD2+, CD15+, and CD96+). In this way, the method advantageously allows for discriminating LSC from HSC. In this respect, a minus means absence of expression or lower expression as compared to a suitable reference, a plus means presence of expression or higher expression as compared to a suitable reference.

A suitable reference for CD34, CD38, and CD45 expression is a reference associated with expression of said markers on (e.g. B, T) lymphocytes (which are CD34−CD38+CD45++). This can be lymphocytes of healthy subject(s), but preferably are lymphocytes of the patient for who the method is carried out (within the obtained sample). For example, lower expression of CD38, CD45 can be determined if the measured expression is at least 5, 10, 20, or 40% lower as compared to this reference. Higher expression of CD34 can be determined if the measured expression is at least 5, 10, 20, or 40% higher as compared to this reference. Alternatively, a reference can be used that is associated with expression of CD34, CD38, and CD45 on confirmed HSC of patients having AML (preferably the patient for who the method is carried out), or, less preferred, HSC of healthy subjects. However, in this alternative scenario the measured expression should be substantially on the same level to indicate lowered expression of CD38 and higher expression of CD34 and CD45, since these HSC are CD34+CD38−CD45+ themselves.

A suitable reference for CD123, CD33, CLL-1, TIM-3, CD11b, CD22, CD45ra, CD44, CD56, CD7, CD2, CD15, and CD96 expression is a reference associated with expression of said markers on (e.g. B, T) lymphocytes (which are substantially negative for these markers). This can be lymphocytes of healthy subject(s), but preferably are lymphocytes of the patient for who the method is carried out. For example, higher expression of a marker can be determined if the measured expression is at least 5, 10, 20, or 40% higher as compared to this reference. However, more preferably, a reference is used that is associated with marker expression on confirmed benign hematopoietic stem cells of patients having AML (preferably the patient for who the method is carried out), or, less preferred, hematopoietic stem cells of healthy subjects. Also in this case, higher expression of a marker can be determined if the measured expression is at least 5, 10, 20, or 40% higher as compared to this reference.

The present method is preferably used during follow-up, but can also be used at diagnosis for subjects having or suspected of having AML, wherein positive identification (presence) of LSC is indicative of indeed having AML. In this regard, a subject suspected as having AML is considered as a subject who has, according to assessment of an appropriate medical practitioner, a non-negligible chance of having AML, for example based on symptoms. In practice, the present method does not necessarily involve a diagnostic method practiced on the human or animal body, nor does it involve treatment of the human or animal body by therapy or surgery.

During disease progression (follow-up) and/or after diagnosis or remission, the present method is very well suited for predicting response to therapy and/or chance of relapse of AML. In such embodiment, the steps of the method are carried out, after which, for the provided sample, the quantity of LSC can be determined, preferably relative to the quantity of white blood cells, or the quantity of blast cells, or the total quantity of stem cells, or the quantity of HSC. In this case the (relative) amount of LSC positively correlates with the chance of relapse of AML.

The present method is well suited to be carried out by flow cytometry. Flow cytometry generally can be seen as a laser-based, biophysical technology employed in cell counting, cell sorting, biomarker detection and protein engineering, by suspending cells in a stream of fluid and passing them through an electronic detection apparatus. It allows simultaneous multiparametric analysis of the physical and chemical characteristics of up to thousands of particles (e.g. cells) per second (FACS analysis). Flow cytometry is routinely used in the diagnosis of health disorders, especially blood cancers, but has many other applications in basic research, clinical practice and clinical trials. Fluorescence-activated cell sorting (FACS sorting) is a specialized type of flow cytometry. It provides a method for sorting a heterogeneous mixture of biological cells, such as including LSC and HSC, into two or more containers, one cell at a time, based upon the fluorescent characteristics (based on presence/abundance or absence of fluorescein-labelled antibodies bound to cell surface markers) and/or specific light scattering of each cell. Fluorophores that can be used as labels in flow cytometry can for example be seen in Table 3. The skilled person knows how to acquire a flow cytometry apparatus such as FACS Canto or FACS Fortessa and FACS Aria (Becton Dickinson, San Jose, Calif., USA) equipped to carry out FACS analysis and/or FACS sorting.

Accordingly, step (b) of the method can be performed by flow cytometry, preferably wherein step (b) further comprises measuring, for individual cells of the provided sample, forward scatter and/or sideward scatter. Increased forward scatter (e.g. at least 10, 20%) and/or increased sideward scatter (e.g. at least 10, 20%) in comparison to a suitable reference, such as (B, T) lymphocytes of the particular patient, confirmed benign hematopoietic stem cells, or hematopoietic stem cells or lymphocytes of healthy subjects, is a further indicator that an individual cell is Leukemic, and absence of said increased forward scatter and/or increased sideward scatter is an indicator that an individual cell is benign.

The method can further comprise an additional step (d) of separating LSC from HSC, which can be carried out by flow cytometric fluorescence-activated cell sorting (FACS sorting) as referred to above.

As a further matter, the present disclosure also overcomes significant obstacles in current methods for screening for new therapeutics targeting LSC. In the current methods, a comparison is made between the effect of individual or combinations of compounds on Leukemic stem cells on the one hand, and on benign (healthy) hematopoietic stem cells on the other hand. However, the control in the form of benign (healthy) hematopoietic stem cells is obtained from healthy volunteers. This is not ideal since increasing insight has led to the notion that those healthy controls are not adequate: the microenvironment in the bone marrow at diagnosis of AML is quite different from that in a healthy bone marrow and this severely affects the properties of the benign hematopoietic stem cells in AML patients. Even after treatment, in the absence of abundant tumour mass, the microenvironment may still be different from that in healthy individuals.

The present disclosure overcomes this problem by comparing effects of putative therapeutics towards Leukemic stem cells and benign hematopoietic stem cells obtained from the same bone marrow from the same AML patient.

In this regard, there is provided for a method for screening for compounds that are able to reduce viability (and/or clonogenic ability and/or engraftment) of cancer stem cells (preferably LSC) and not, or to a lesser extent, viability (and/or clonogenic ability and/or engraftment in a mouse model) of benign stem cells (preferably HSC), the method comprising
(a) providing LSCs and HSCs obtainable according to the present disclosure;
(b) providing at least one test compound;
(c) measuring change in viability (and/or clonogenic ability and/or engraftment) of (at least part of) the LSCs of step (a) after contacting said (at least part of) LSCs with said at least one compound,
(d) measuring change in viability (and/or clonogenic ability and/or engraftment) of (at least part of) the HSCs of step (a) after contacting said (at least part of) HSCs with said at least one compound.

If reduced viability (and/or clonogenic ability and/or engraftment) is measured in step (c) and no or less reduced viability (and/or clonogenic ability and/or engraftment) is measured in step (d), this indicates that the at least one compound is able to reduce cancer stem cell (preferably LSC) viability (and/or clonogenic ability and/or engraftment) and not, or to a lesser extent, benign stem cell (preferably HSC) viability (and/or clonogenic ability and/or engraftment). Such compound could be a promising new therapeutic. Steps (c) and/or (d) can be performed in a mouse model, wherein for step (c) LSC can be introduced in a mouse as described in further detail elsewhere herein, and for step (d) HSC can be introduced in a mouse as also described in further detail elsewhere herein. Alternatively, a mouse model can be used to confirm that viability (and/or clonogenic ability and/or engraftment) of LSC contacted with the compound is indeed reduced. Clonogenic ability can be understood as the ability of a cell to proliferate indefinitely.

It is noted that a Leukemic bone marrow generally always contains a remnant of normal healthy stem cells. To date it was however not possible to adequately prospectively trace HSC due to the fact that they are functionally and immunophenotypically similar to LSC. Identification and study of the co-existing LSC and HSC in one bone marrow sample offers a much better model for new specific target finding than the comparison of LSC versus the HSC isolated from normal bone marrow. In this way, new anti-LSC therapies can be developed that confer specific eradication of the Leukemic stem cells while sparing the benign (normal) hematopoietic stem cell.

The present disclosure further provides a container (e.g. a (FACS) tube) that can be used for performing the present method. This container comprises a set of at most 25, 24, 23, 22, 21, 20, 19, 18, 17, 16, 15, 14 or 13 antibodies, including antibodies against CD34, CD38, CD45 CD123, CD33, CLL-1, TIM-3, CD11b, CD22, and preferably CD45ra and/or CD44. Preferably, the set also includes antibodies against CD56 and/or CD7. Further, antibodies against CD2, CD15, and/or CD96 may be included in the set.

Further explanation of characteristics or features of the markers and/or antibodies disclosed herein, including how they can be obtained, can for example be found in Zeijlemaker and Schuurhuis (Chapter 6 in Leukemia, Guenova, ed., ISBN: 978-953-51-1127-6, InTech, 2013), and more specifically for CD45, and CD45ra in Crosbie et al Hepatology Vol. 29(4) 2003; for CD34 in Bachas et al Leukemia 26(6), 1313-20, 2012; for CD38 in Costello et al Cancer Res 60(16) 4403-11, 2000; for CD123 in Jordan et al Leukemia 14(10) 1777-84, 2000; for CD33, CD11b, CD22, CD56, CD7, CD2, and CD15 in van Rhenen et al Leukemia 21(8) 1700-7, 2007; for CLL-1 in van Rhenen et al Blood 110(7) 2659-66, 2007; for TIM-3 in Jan et al Proc NAtl Acad Sci USA 108(12) 5009-14, 2011; for CD44 in Jin et al Nat Med 12(10) 1167-74, 2006; and for CD96 in Hosen et al Proc Natl Acad Sci USA 104(26), 11008-13, 2007.

Of course, in practice, the container can comprise a sample of a subject, preferably a bone marrow sample or a peripheral blood sample.

Advantageously, the antibodies against CLL-1, TIM-3, CD11b, CD22, and preferably also CD7, CD56 can be provided with the same detection label (fluorescein, preferably PE), so that they fit into one fluorescence channel.

Finally the present disclosure also provides for a kit suitable for performing the method according to the present disclosure, wherein the kit comprises at least one container as described above.

As disclosed herein, aberrant marker expression, preferably in combination with (other) flow cytometric parameters can advantageously be used to discriminate HSC from LSC in CD34+CD38− AML cases. The term marker as used herein particularly refers to cell surface markers which are proteins expressed on the surface of cells that can conveniently serve as markers of specific cell types, such as LSC or HSC.

Cell surface marker expression (i.e. presence and/or abundance of the marker on the cell surface) on the total blast compartment and the CD34+CD38-cells thereof can be measured as previously described (e.g. van Rhenen et al 2007 Blood 110:2659-2666; Feller et al 2004 Leukemia 18:1380-1390). Purified white blood cells can be obtained from a (frozen-thawed) bone marrow sample or peripheral blood sample using lysing solution (e.g. Pharm lyse, BD Biosciences) to eradicate red blood cells. After washing with PBS containing 0.1% human serum albumin (HSA), cells can be re-suspended in PBS containing 0.1% HSA, incubated with monoclonal antibody combinations (mAbs) against the set of markers (available for example from Becton Dickinson) for e.g. 15 minutes at room temperature and can then be washed with PBS containing 0.1% HSA. Analysis of marker expression of the CD34+CD38− can be done for example as described by van Rhenen et al (van Rhenen et al 2007 Leukemia 21:1700-1707). Samples can be analysed for example using a FACS Canto from Becton Dickinson (BD, San Jose, Calif., USA) using FACS-DIVA or Infinicyt software.

Identifying CD34+CD38− cells can performed for example with the following combination of antibodies: CD34 BV421 (clone 581), Becton Dickinson, BD), CD45 V500c (clone 2D1, BD) and CD38 APC (clone HB7, dilution 1:50, BD). For identifying LSC, the following combination of antibodies can be used: TIM-3 PE (clone 344823, dilution 1:10, R&D Systems), CD7 PE (clone M-T701, dilution 1:20, BD), CD11b PE (clone D12, dilution 1:200, BD), CD56 PE (clone MY31, dilution 1:50, BD), CD22 PE (clone S-HCL-1, BD), and CLL-1 PE (clone 50C1, BD) (see also van Rhenen et al 2007 Blood 110:2659-2666) (preferably these antibodies are PE labelled), as well as antibodies CD123 PerCP-CY5.5 (clone 7G3, BD), CD44 APC-H7 (clone 644/26, dilution 1:50, Beckman Coulter), CD33 (clone P67.6, BD), and CD45ra (clone L48, BD). Optionally, further antibodies CD2 (clone MT910, BD), CD15 (clone MMA, dilution 1:100, BD) and CD96 (clone 6F9, dilution 1:10, BD) can be used. Forward scatter (FSC, reflecting cell size) and sideward scatter (SSC, reflecting cell granularity) can be measured as described in for example Harada et al 1994 (J. Cancer Res. Clin. Oncol. 120:553-557).

Confirmed benign HSC can be distinguished from LSC for example by injection into highly immune deficient mice. If LSCs are injected, this leads human Leukemic engraftment, while if HSCs are injected, this leads to human multilineage engraftment (see also Pearce et al 2006 Blood 107:1166-1173; Yahata et al 2003 Blood 101:2905-2913). To do this, NOD/SCID IL-2Rγ −/− mice can be obtained from the Jackson laboratory (Bar Harbor, Me., USA). Mice, at the age of 8-10 weeks, which can be irradiated sub-lethally with a dose of

350 cGy, 24 hours prior to transplantation of the human AML cells. Mice can be evaluated for human AML engraftment after a maximum of 16 weeks, or earlier when becoming ill (hunch-back, substantial weight loss and a ruffled coat). Samples that initiate human leukaemia engraftment, can be selected for subsequent experiments. Cell fractions from these samples can be injected intrafemorally. The method of intrafemoral injection can for example be adapted from Yahata et al., but in addition a 27G needle can be used to make a small hole in the femur, and subsequently the cells are injected using an insulin syringe with a fixed 30G needle. Cells in PBS/0.1% HSA in a volume up to 30 RI can be injected into the bone.

Injections can be performed under complete anaesthesia (250 RI of a ketamin 10 mg/ml/xylazin 1 mg/ml mix) and analgesia was given subcutaneously (Carprofen 4 mg/kg). Mice are kept for a maximum of 16 weeks after injection of the human cells, after which they were sacrificed and analysed for engraftment. In case of illness, mice can be sacrificed earlier. The screening for human cells in mouse bone marrow can be done using flow cytometry with a human PerCp labelled CD45 monoclonal antibody (clone 2D1, dilution 1:20, BD) and a murine PE-labelled antibody (clone 30-F11, dilution 1:2,000, BD-Pharming). Human leukemic engraftment can be determined based on positivity for CD45-PercP, the absence of CD19-positive B cells and the presence of for example CD33 positive cells. Human multilineage engraftment can be identified when CD45 positive cells are identified, that consist of both CD19 positive B-cells and myeloid cells with for example CD33 present. In addition, in the case of molecular aberrancies, specific FISH or PCR analysis can be performed on engrafted cells, which for certain AML cases can be performed for example as detailed below.

Another way to identify confirmed benign HSC (from LSC) is PCR, optionally with FISH analysis. For the FISH analysis, cytospins can be prepared with cells sorted according to the present disclosure. LSI AML1/ETO dual color for t(8;21) probe (Vysis) can be applied to the denatured cells and incubated as previously described (van Rhenen et al 2007 Leukemia 21:1700-1707). Genomic DNA from sorted cell populations can be analysed for the presence of an FLT3-ITD as described before (Cloos et al 2006 Leukemia 21:1217-1220). Mutations in NPM1exon 12 can be analyzed via PCR on genomic DNA that is isolated from the sorted cell fractions. PCR amplification can be subsequently performed with the following primers: NPM1 forward: 5′-TTAACTCTCTGGT-GGTAGAATGA-3′ (SEQ ID NO:1); NPM1 reverse: 5′-CTGACCACCGCTACTACTATGT-3′ (SEQ ID NO:2), located in intron 11 and exon 12, respectively. Subsequent fragment analysis can be performed with a tetrachlorofluorescein phosphoramidite-labeled (Biolegio, Nijmegen, The Netherlands) forward primer. Presence of the mutation(s) indicates LSC, absence confirms benign HSC. Mutations detected with melting curve analysis can further be confirmed by bidirectional DNA sequencing on an ABI 3500 automated sequencer with the use of the BigDye terminator kit (Applied Biosystems Inc). For both FLT3 and NPM1 analysis, the bulk of AML blasts (CD34+CD38+, or with lower CD34 percentages, the CD45^{dim} fraction) can be used as an internal positive control, while lymphocytes can serve as an internal negative control.

### BRIEF DESCRIPTION OF THE FIGURES

FIG. 1: Overview of score per marker according to the scoring system of Table 1.

FIG. 2: Marker stability on HSCs. Ratios of the intensity of marker expression are presented as Median Fluorescence Intensity (MFI) of CD34+CD38− normal stem cells divided by MFI of control population, ie lymphocytes. A comparison is shown between HSCs in normal bone marrow (NBM, the first of the three columns for each marker), HSCs present at AML diagnosis (the second column for each marker) and HSCs in regenerating bone marrow (the third column for each marker). Since the control population of lymphocytes have less autofluorescence than HSCs, the MFI ratio is usually >1. At follow-up, the MFI ratio is slightly higher than control BM. Considerable up-regulation is seen on HSCs in follow-up samples for CD123, while CD33 expression is already high in normal BM. Box plots represent median values and upper (75%) and lower (25%) quartiles. Outliers are plotted as individual points: filled circles represent mild outliers and stars extreme outliers.

FIG. 3: Overview of the best markers (CD33 and Cd123 excluded) in 56 AML samples where only one optimal LSC marker was present.

FIG. 4: Prognostic value of LSC frequency at AML diagnosis. In dependence of the LSC frequency (>0.03% or ≦0.03%), the fraction of patients who, after having entered remission of the disease, experience a period free of disease (relapse free survival, RFS) is shown.

FIG. 5: Prognostic value of LSC frequency after the first cycle (A), and second cycle (B) of chemotherapy and after consolidation therapy (C). Shown is RFS for patient groups with high (>4 in a million white blood cells) or low (≦4 in a million white blood cells) numbers of LSC.

The following Examples illustrate the different embodiments of the disclosure. Unless stated otherwise all techniques are carried out according to standard protocols as described in e.g. Sambrook et al. (1989) Molecular Cloning: A Laboratory Manual, Second Edition, Cold Spring Harbor Laboratory Press; and Sambrook and Russell (2001) Molecular Cloning: A Laboratory Manual, Third Edition, Cold Spring Harbor Laboratory Press, NY; and in Volumes 1 and 2 of Ausubel et al. (1994) Current Protocols in Molecular Biology, Current Protocols, USA.

### Example

The present inventors aimed to create a tool that detects LSCs and HSCs at diagnosis and follow up that takes into account disappearing and newly forming clones. Moreover, this tool should be broadly applicable in a multi-institutional setting, relatively cheap and less time—and bone marrow consuming compared to current detection methods. Ideally, the tool should be applicable as a single multi-parameter stem cell tube for use in FACS flow cytometry (currently up to seven tubes are necessary).

To decide which markers to use for that tube, the present inventors have established expression patterns of the following stem cell markers in 249 AML patients at diagnosis: CLL-1 (also referred to as Clec12a), TIM-3, CD2, CD7, CD11b, CD13, CD14, CD15, CD19, CD22, CD33, CD44, CD44v6, CD47, CD56, CD96 and CD123.

AML samples were considered evaluable when at least 5 stem cells were present in the CD34+CD38− compartment. This was the case in 209/249 cases. In 67/209 cases only HSCs were present in the stem cell compartment. This compartment thereby offered the perfect negative control. As a result, analyses were performed on the remaining 142 cases with LSCs (and in most cases also HSCs) present at diagnosis.

Since the inventors were not able to detect a consistent pattern of CD44v6 and CD47 expression on LSCs (often also expressed on HSCs), these markers have been omitted from further analyses. In general a marker was considered present on LSCs when it scored 1, 2 or 3 points according to the following scoring system:

TABLE 1
Scoring System Leukemic Stem Cell Marker Expression

0 points: no (clear) distinction between LSCs and HSCs
and
no useful expression on LSCs*
1 point: (clear) distinction between LSCs and HSCs
or
useful expression*
2 points: (clear) distinction between LSCs and HSCs
and
no pollution of marker negative fraction# or useful expression*
3 points: (clear) distinction between LSCs and HSCs
and
no pollution of marker negative fraction#
and
useful expression*

All scoring is done by defining for each AML case the best marker and to compare the performance of another marker with this best marker for the AML case under consideration. The best marker has the highest expression of LSC and the best separation between confirmed LSC and HSC.

* Definition of useful expression: expression of the marker on LSCs in CD34+CD38− compartment is 50% or less different as compared to the best marker for the AML case under consideration.
# Definition of pollution: Marker negative LSCs that are present in the marker negative HSC compartment. If there is more than 10% difference between marker negative CD34+CD38− fraction of the marker (as % of total CD34+CD38− compartment) and marker negative CD34+CD38− fraction of the best marker in that particular patient. In other words, Marker negative LSCs that are present in the marker negative HSC compartment as determined using the fact that marker negative LSC, similar to marker positive LSC may have higher FSC/SSC than HSC.

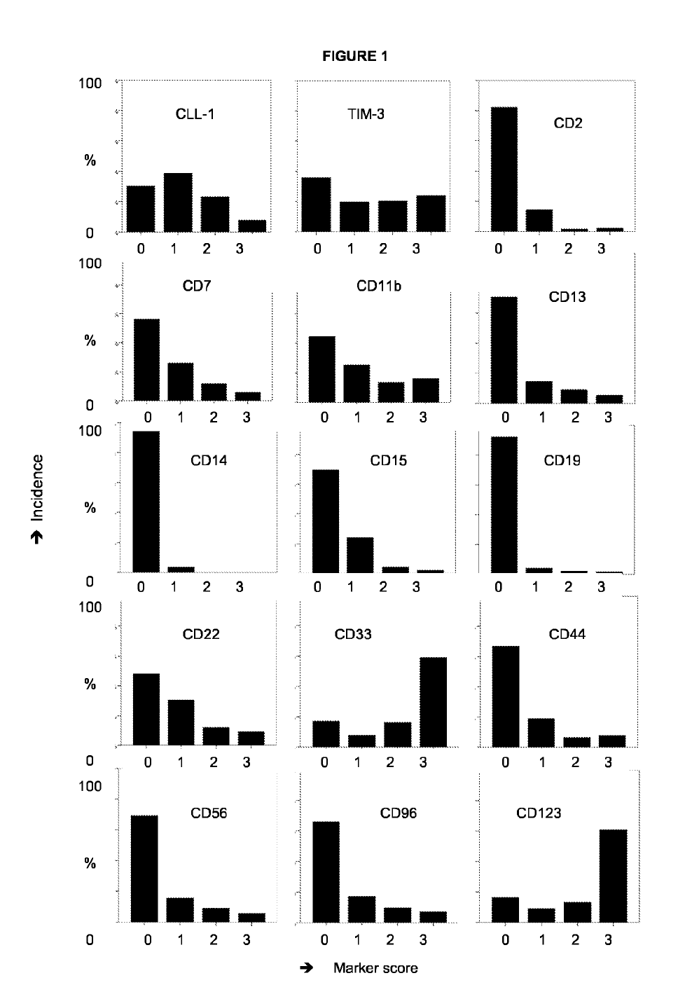

The distribution of different scores were quite different among the patients for the markers studied, as shown in FIG. 1. A summary of this is shown as prevalence of marker expression (irrespective their score, i.e. 1, 2 or 3) in Table 2:

TABLE 2

| | Number of cases with |
|---|---|
| | marker present on LSC |
| Marker | (%) |
| CD123 | 83.7 |
| CD33 | 83.0 |
| CLL-1 | 69.7 |
| TIM-3 | 64.1 |
| CD11b | 55.3 |
| CD22 | 51.8 |
| CD7 | 43.7 |
| CD44 | 35.9 |
| CD96 | 34.0 |
| CD56 | 30.7 |
| CD15 | 30.5 |
| CD13 | 29.0 |
| CD2 | 17.7 |
| CD19 | 7.8 |
| CD14 | 3.5 |

CD123 and CD33 have the highest incidence in this set of patients (Table 2) and, moreover, have the most favorable distribution of scores (compared to other markers, CD123 and CD33 have high numbers of “3” scores in FIG. 1).

However, the results presented in Table 2 and FIG. 1 show that potentially the number of usable LSC markers exceeds the number of fluorescence channels available for flow cytometry: 15 different LSC markers. With a backbone of CD34, CD38 and CD45, there are 18 markers in total. To be able to define a single stem cell tube, the present inventors came up with the idea to combine markers in the same fluorescence channel. This may only be possible if markers in that fluorescence channel have no, or very low, expression on HSCs, both at diagnosis and follow up (otherwise HSC may falsely be scored as LSC).

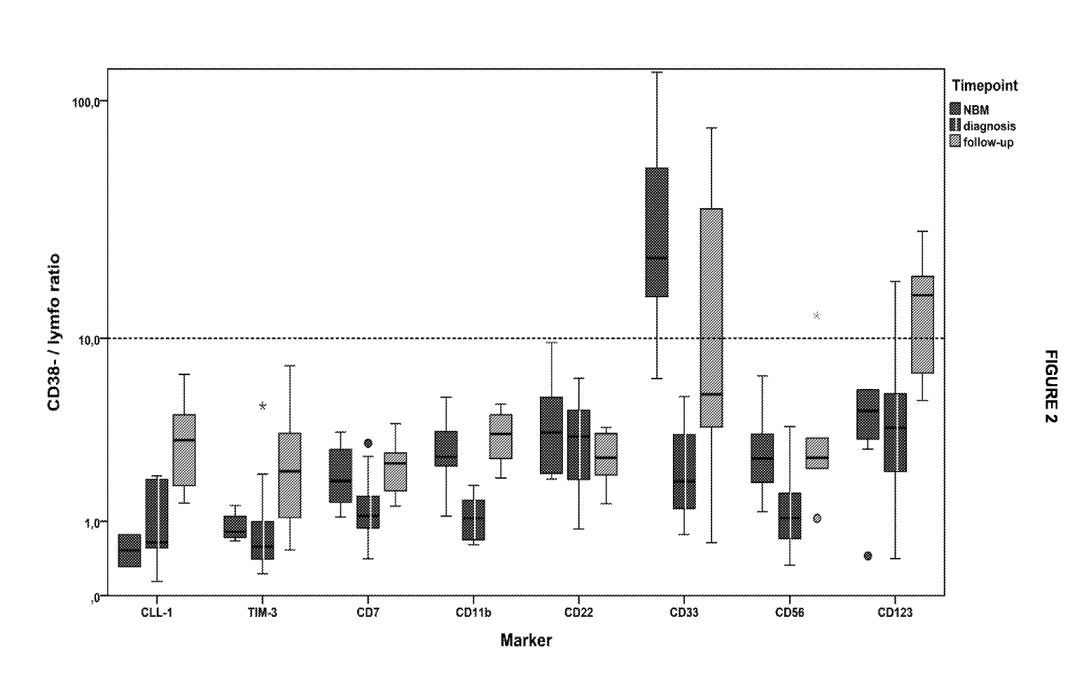

Most markers of Table 2 fulfill the condition that there is no, or very low, expression on HSCs. However, CD33 and CD123 do not (FIG. 2). Since expression of these two markers on AML LSC is usually much higher than on HSC, while in a considerable part of the cases, these two even represent the best marker (FIG. 1, Table 2), CD33 and CD123 were nevertheless chosen in the panel, although, due to expression on HSC, not to be combined with any of the other markers in the same fluorescence channel. CD13 (not shown in FIG. 2) was excluded based on its variable expression on HSCs (data not shown) and on the relatively low prevalence on LSC (FIG. 1, Table 2). Finally, CD44 (also not shown) was included as it had a very high expression on HSC that nevertheless may be different from LSC.

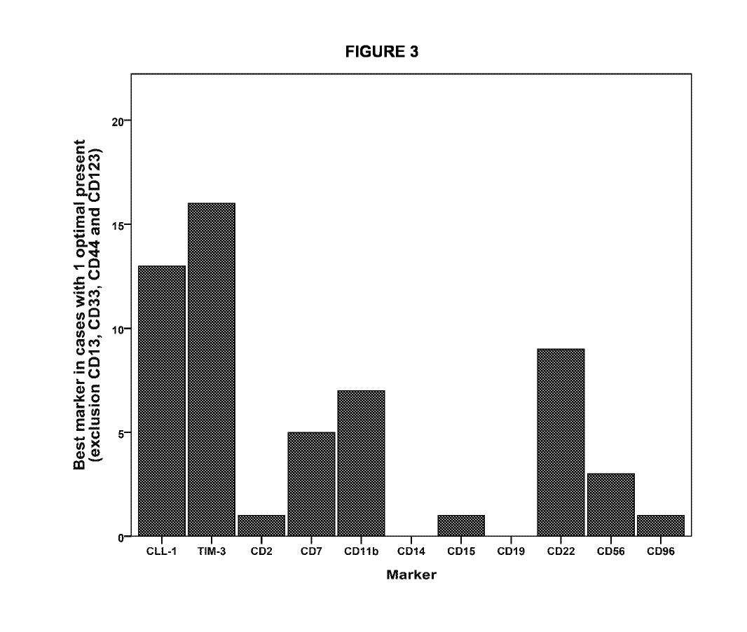

In particular because CD13, CD33, CD44, and CD123 can be positive on HSCs and are therefore not suitable to be used together with other markers in the PE channel, the inventors then explored the possibility to use antibodies against the remaining LSC markers (CLL-1, TIM-3, CD2, CD7, CD11b, CD14, CD15, CD19, CD22, CD56, and CD96) together in the PE-channel. First, it was studied if redundancy for these markers would allow to reduce the number. To that end focus was on 56/142 AML cases where, according to the scoring system shown in Table 1, there was only 1 best marker present, which would thereby suggest that such marker should be included in the PE channel of the single tube. FIG. 3 shows that CLL-1, TIM-3, CD7, CD11b, CD22 and CD56 are preferred markers to combine in the PE channel. FIG. 3 also demonstrates that CD2, CD15 and CD96 each are optimal markers in 1/56 (1.8%) of the cases, since their general presence on LSCs is not that high (FIG. 1, Table 2), while, in addition, CD123 and CD33 also offered relevant information in these cases. Based on this, we have concluded that the additive value of these three markers is limited. As a consequence we have decided to not include these three markers in the detection tube. In the remaining 86/142 AML cases there are ≧2 best markers present, and these always include CLL-1 and/or TIM-3 and/or CD7 and/or CD11b and/or CD22 and/or CD56 (data not shown).

The next issue was to verify the stability of these 6 markers, or in other words, verify if these 6 markers which expression generally is low/negative on HSC at diagnosis would not be up-regulated during follow up. FIG. 2 shows these markers fulfill this property.

CD45RA can be included in the tube in the FITC channel, based on our observations that CD45RA identifies two types of AML that differ in their CD34+CD38− LSC populations. In one type LSCs have identical scatter properties as the corresponding HSC and in the other type LSCs have higher scatter than the HSC. This may translate to different survival, but CD45RA anyhow additionally helps to define HSC (always CD45RA negative) and LSC (in part of AML cases, all CD45RA+).

CD44 is a marker that can be used for the detection of LSCs (Table 2). However, expression on both LSC and HSC is very high, and, similar to CD123 and CD33, expression patterns of these antigens have to be analyzed individually. Main reason to include CD44 is the possibility to define nonspecific events that pop up at fluorescence values that are far below the specific LSC and HSC events that are extremely high. This information can subsequently be used to identify nonspecificity in the other fluorescence channels, e.g. via their identification in CD34, CD45, CD38 and FSC/SSC defined plots.

Based on the issues of stability and redundancy, identification of nonspecific staining and differentiation stages in the CD34+CD38− LSC, the LSC panel can most preferably be designed as presented in Table 3:

TABLE 3

| Fluorescence | | | | | | | | |
|---|---|---|---|---|---|---|---|---|
| channel | FITC | PE | PerCP-CY5.5 | PeCy7 | APC | APC-H7 | BV421 | V500c |
| Markers | CD45ra | CLL-1 | CD123 | CD33 | CD38 | CD44 | CD34 | CD45 |
| | | TIM-3 | | | | | | |
| | | CD7 | | | | | | |
| | | CD11b | | | | | | |
| | | CD22 | | | | | | |
| | | CD56 | | | | | | |

A major advantage of the approach to incorporate redundancy of markers is that room remains to include new/additional stem cell markers in the PE-channel. As previously mentioned, it is of importance that such (new) markers will not be expressed on HSCs throughout the disease and treatment.

Besides using the PE-marker combination, CD33, CD123, CD45RA and CD44, the specificity of the LSC detection can be further improved via the use of secondary gating strategies. In approximately 50% of the CD34 positive AML patients these secondary parameters can be applied making use of the fact that LSCs and HSCs may differ in forward scatter (FSC, reflecting cell size), and/or sideward scatter (SSC, reflecting cell granularity). In particular, LSC may have at least 10% increased forward scatter ratios and/or at least 10% increased sideward scatter ratios, in comparison to HSC.

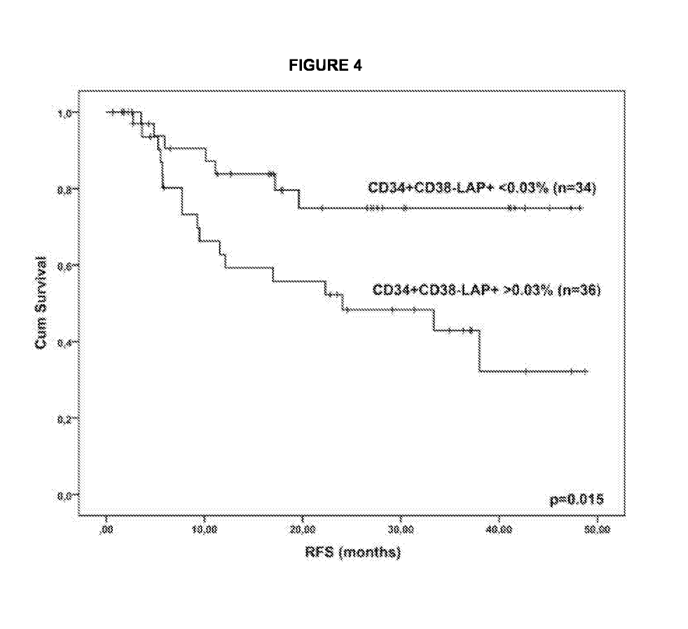

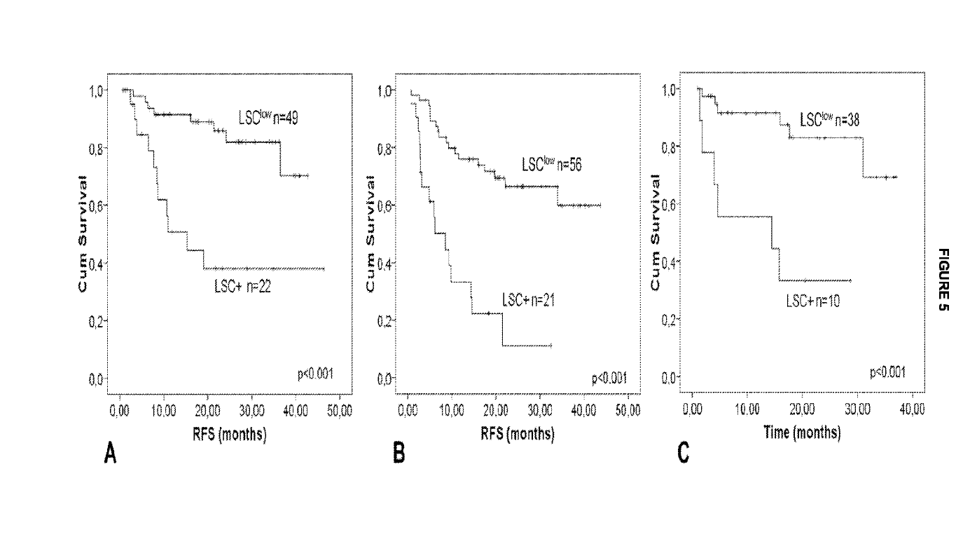

It was found that with the presently described LSC detection tube, wherein the fluorescence on stem cells of CLL-1, TIM-3, CD7, CD11b, CD22 and CD56 are added together in the PE-channel, the real frequency of the LSC population could be established more accurately as compared to the single markers in single channels as previously used. Moreover, this can be achieved in a less time—and material consuming manner. FIGS. 4 and 5 show that higher LSC frequency in a diagnosis or a remission bone marrow, respectively, predicts relapse in AML patients.

1. Method for identifying Leukemic stem cells (LSC), the method comprising:
(a) providing a sample obtained from a subject having or suspected of having Acute Myeloid Leukemia or Myelodysplastic Syndrome;
(b) measuring, for individual cells of the sample, expression of a set of at most 20 markers, the set comprising markers CD34, CD38, CD45, CD123, CD33, CLL-1, TIM-3, CD11b, and CD22;
wherein an individual cell is identified as a Leukemic stem cell if the cell is CD34+, CD38−, CD45+, and at least one of CD123+, CD33+, CLL-1+, TIM-3+, CD11b+, and CD22+.

2. Method according to claim 1, wherein the set of markers comprises at most 19, 18, 17, 16, 15, 14, or 13 markers.

3. Method according to claim 1, wherein the set of markers further comprises markers CD45ra and/or CD44.

4. Method according to claim 1, wherein the set of markers further comprises markers CD56 and/or CD7.

5. Method according to claim 1, wherein step (b) is performed by flow cytometry, and preferably wherein step (b) further comprises measuring, for individual cells of the provided sample, forward scatter and/or sideward scatter.

6. Method according to claim 1, wherein the method further comprises step (c) of separating LSC from benign hematopoietic stem cells (HSC), preferably by flow cytometric fluorescence-activated cell sorting.

7. Method according to claim 1, wherein the sample is a bone marrow sample or a peripheral blood sample.

8. Method according to claim 1, wherein the method is performed during diagnosis and/or during follow-up.

9. Method for predicting response to therapy and/or chance of relapse of Acute Meyeloid Leukemia, the method comprising:
(a) performing the steps according to claim 1,
(b) after step (a), determining, for the provided sample, the quantity of LSC.

10. Method for screening for compounds that are able to reduce viability and/or clonogenic ability and/or engraftment of cancer stem cells and not, or to a lesser extent, viability and/or clonogenic ability and/or engraftment of benign stem cells, the method comprising:
(a) providing LSCs and HSCs obtainable by the method according to claim 1,
(b) providing at least one compound,
(c) measuring change in viability and/or clonogenic ability and/or engraftment of the LSCs of step (a) after contacting said LSCs with said at least one compound,
(d) measuring change in viability and/or clonogenic ability and/or engraftment of the HSCs of step (a) after contacting said HSCs with said at least one compound.

11. Container comprising a set of at most 20 antibodies, the set comprising antibodies against CD34, CD38, CD45 CD123, CD33, CLL-1, TIM-3, CD11b, CD22, and preferably CD45ra and/or CD44.

12. Container according to claim 11, wherein the set of antibodies comprises at most 19, 18, 17, 16, 15, 14, or 13 antibodies.

13. Container according to claim 11, wherein the set of antibodies further comprises antibodies against CD56 and/or CD7.

14. Container according to claim 11, wherein the antibodies against CLL-1, TIM-3, CD11b, CD22 are provided with the same detection label.

15. Container according to claim 11, further comprising a sample of a subject, preferably a bone marrow sample or a peripheral blood sample.
